(19) United States
(12) Patent Application Publication
(10) Pub. No.: US 2017/0050000 A1
(43) Pub. Date: Feb. 23, 2017

(54) ANCHORED GUIDEWIRE WITH MARKINGS TO FACILITATE ALIGNMENT

(71) Applicant: CLEARSTREAM TECHNOLOGIES LIMITED, Enniscorthy, County Wexford (IE)

(72) Inventor: Michael Adam RANDALL, Tempe, AZ (US)

(21) Appl. No.: 15/118,512

(22) PCT Filed: Feb. 17, 2015
(86) PCT No.: PCT/US2015/016155

§ 371 Date: Aug. 12, 2016

Related U.S. Application Data
(60) Provisional application No. 61/940,675, filed on Feb. 17, 2014.

(51) Int. Cl. A61M 25/09; A61F 2/95; A61M 25/01; A61B 17/22; A61M 25/10

(52) U.S. Cl. CPC A61M 25/09; A61B 17/22; A61M 25/10; A61M 25/104; A61M 25/0108; A61F 2/95; A61M 2025/09125; A61M 2025/09166; A61B 2017/22001; A61B 2017/22047; A61B 2017/22062; A61B 2017/22042; A61M 2025/105; A61M 2025/1063

(57) ABSTRACT

An apparatus for treating a treatment area in the vasculature using a catheter, such as one designed to deliver a payload (such as a therapeutic agent (e.g., a drug, such as paclitaxel, rapamycin, heparin and the like), a stent, a stent graft, or a combination thereof) or a working element (such as a cutter, focused force wire, or the like) to a specified location within the vasculature. The catheter may include at least one first radiopaque marking. A guidewire (26) for guiding the catheter to the treatment area includes an anchor (26a) for selectively anchoring the guidewire to the vasculature, and at least one second radiopaque marking (26b) for corresponding to the at least one first radiopaque marking of the catheter when positioned at the treatment area.

This application claims the benefit of U.S. Provisional Patent Application Ser. No. 61/940,675, the disclosure of which is incorporated herein by reference.

### TECHNICAL FIELD

This disclosure relates generally to interventional medical devices and procedures, such as angioplasty using a balloon catheter, and, more particularly, to an anchored guidewire with markings to facilitate the precise location of a treatment at a treatment area.

### BACKGROUND

Catheters including balloons are routinely used to resolve or address flow restrictions or perhaps even complete blockages in tubular areas of a body, such as arteries or veins. In many clinical situations, the restrictions are caused by hard solids, such as calcified plaque, and may sometimes involve the use of high pressures to compact such blockages. Commercially available balloons employ complex technology to achieve high pressure requirements without sacrificing the profile of the balloon. Besides high pressure requirements, the balloons should also be resistant to puncture, easy to track and push, and present a low profile, especially when used for angioplasty.

The clinician performing the angioplasty procedure should be able to locate the position of the uninflated balloon with accuracy, so that the balloon will be properly positioned once inflated. This is conventionally accomplished by attaching marker bands to the catheter shaft corresponding to the ends of the balloon working surface (which is the surface along the portion of the balloon typically used to achieve the desired treatment effect, such as contacting the calcified plaque, which surface in the case of a balloon having conical or tapering sections at the proximal and distal ends is typically co-extensive with a generally cylindrical barrel section).

Misalignment of the marker bands during placement along the shaft sometimes results in their failure to correspond precisely to the extent of the working surface. This misalignment may prevent the clinician from accurately identifying the location of the working surface of the balloon during an interventional procedure. Also, when successive intravascular interventions are made, such as during a pre-dilatation using a first catheter followed by dilatation using a second catheter, the clinician must guess at the location where the pre-dilatation occurred. In either case, this uncertainty may lead to a geographic misalignment, or “miss,” of the desired contact between the intended treatment area and the working surface of the balloon. It is especially desirable to avoid such an outcome when the balloon is designed to deliver a payload (such as a therapeutic agent (e.g., a drug, such as paclitaxel, rapamycin, heparin and the like), a stent, a stent graft, or a combination thereof) or a working element (such as a cutter, focused force wire, or the like) to a specified location within the vasculature, since a miss may, at a minimum, prolong the procedure (such as, for example, by requiring redeployment of the balloon or the use of another balloon catheter in the case of a drug coated balloon), and possibly result in an inferior outcome if the lesion is not properly treated as a result of the misalignment.

In order to assess the length of a lesion from a location external to the body, a clinician may use an external ruler, which in one form is called a “LeMaitre” tape. While the use of such a ruler or tape may allow for a more precise assessment of the lesion length and an area treated by a pre-diliation step, it is not without limitations. For one, a displacement or difference in the apparent position of the lesion margins results when viewed along two different lines of sight. This “parallax” can lead to an inaccurate measurement and, at a minimum, contribute to the geographic misalignment of the working surface relative to the lesion. The use of such a ruler may also lead to inferior measurements when the vasculature at issue is particularly tortuous.

Accordingly, a need exists for a manner in which to position a catheter including a treatment into the vasculature at a treatment area with enhanced accuracy, and also in a manner that is highly repeatable.

### SUMMARY

An object of the disclosure is to provide a guidewire with an anchoring device and at least one marking visible during the procedure (such as under fluoroscopy) for use in properly aligning a treatment within the vasculature.

In one embodiment, an apparatus for treating a treatment area in the vasculature using a catheter including at least one first radiopaque marking includes a guidewire for guiding the catheter to the treatment area. The guidewire comprises an anchor for selectively anchoring the guidewire to the vasculature, and further includes at least one second radiopaque marking for corresponding to the at least one first radiopaque marking of the catheter when positioned at the treatment area.

In this or another embodiment, the guidewire may include a plurality of second radiopaque markings. The plurality of second radiopaque markings on the guidewire may be equidistantly (or regularly) spaced, or not. The anchor may be selected from the group consisting of a self-expanding stent, a self-expanding braided stent, a barb, a plurality of extensible fingers, and an inflatable balloon.

In one possible embodiment, the anchor comprises an inflatable balloon and the guidewire comprises a lumen for inflating the balloon. The anchor may comprise a shape memory material. The anchor may be provided at a distal end of the guidewire, and the at least one second radiopaque marking may be proximal of the anchor.

According to another aspect of the disclosure, an apparatus for treating a treatment area in the vasculature comprises a catheter including at least one first radiopaque marking and a guidewire for guiding the catheter to the treatment area and including an anchor for anchoring the guidewire to the vasculature. The guidewire includes at least one second radiopaque marking adapted for corresponding to the at least one first radiopaque marking of the catheter when positioned at the treatment area.

The guidewire may include a plurality of second radiopaque markings, which may be equidistantly or irregularly spaced. The anchor may be selected from the group consisting of a self-expanding stent, a self-expanding braided stent, a barb, a plurality of extensible fingers, and an inflatable balloon. In one embodiment, the anchor comprises an inflatable balloon and the guidewire comprises a lumen for inflating the balloon.

The anchor may be located at a distal end of the guidewire, and the at least one second radiopaque marking may be proximal of the anchor. The catheter may include an inflatable balloon, which may include the at least one radiopaque marking. The catheter may comprise a shaft adapted for receiving the guidewire, which shaft may further include the at least one first radiopaque marking for aligning with the at least one second radiopaque marking of the guidewire.

The apparatus may further include a second catheter including an expandable device having a treatment. The treatment may be selected from the group consisting of a drug, a stent, a stent graft, a cutter, a focused force wire, or any combination of the foregoing.

According to a further aspect of the disclosure, a kit is provided for treating a treatment area in the vasculature. The kit comprises a first catheter including at least one first radiopaque marking, a second catheter including at least one second radiopaque marking, and a guidewire for guiding the first or second catheter to the treatment area and including an anchor for anchoring the guidewire to the vasculature. The guidewire includes at least one second radiopaque marking adapted for corresponding to the at least one first radiopaque marking of the first catheter or the at least one second radiopaque marking of the second catheter when positioned at the treatment area.

In any of the foregoing embodiments, an actuating means or actuator may be provided, which may be arranged to actuate the anchor to thereby anchor the guidewire to the vasculature. The anchor may be being arranged so that it will, when introduced into a patient's vasculature, automatically assume a configuration capable of anchoring the guidewire to the patient's vasculature.

According to a further aspect of the disclosure, a method of performing an interventional procedure in the vasculature comprises: (1) removably anchoring a guidewire including at least one first radiopaque marking into the vasculature; and (2) delivering a catheter including a second radiopaque marking along the guidewire to a position where the second radiopaque marking aligns with the first radiopaque marking of the guidewire. The method may further include the steps of removing the first catheter, and delivering a second catheter including a third radiopaque marking along the guidewire to a position where the third radiopaque marking aligns with the first radiopaque marking of the guidewire.

A further aspect of the disclosure pertains to a method of assessing a size of a treatment location in the vasculature. The method comprises positioning a guidewire including a plurality of radiopaque markings adjacent the treatment location, using the markings to assess the size of the treatment location, and delivering a first catheter along the guidewire to the treatment area to a position where at least one radiopaque marking on the catheter aligns with at least one radiopaque marking on the guidewire. The positioning step may comprise anchoring the guidewire in the vasculature, and the anchoring step may comprise deploying an anchor connected to the guidewire distally of the treatment location. The method may further include the step of delivering a second catheter along the guidewire to a position where at least one radiopaque marking on the second catheter aligns with at least one radiopaque marking on the guidewire.

### BRIEF DESCRIPTION OF THE DRAWINGS

FIGS. 1-6 illustrate various features of balloon catheters.

FIGS. 7 and 7a-7c illustrate various embodiments according to the disclosure.

FIGS. 8-10 illustrate one possible use of the guidewire according to the disclosure.

FIGS. 11-12 illustrate a catheter with radiopaque markings, which may be co-located with the guidewire.

### MODES FOR CARRYING OUT THE INVENTION

The description provided below and in regard to the figures applies to all embodiments unless noted otherwise, and features common to each embodiment are similarly shown and numbered.

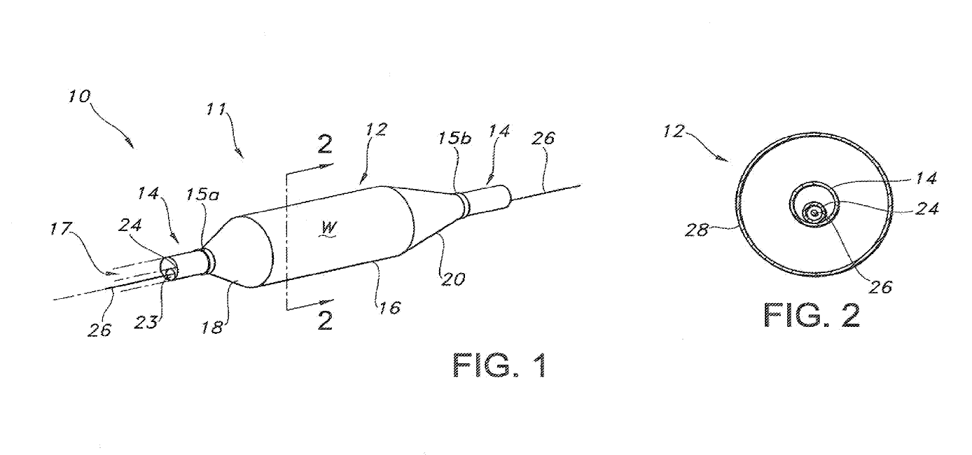

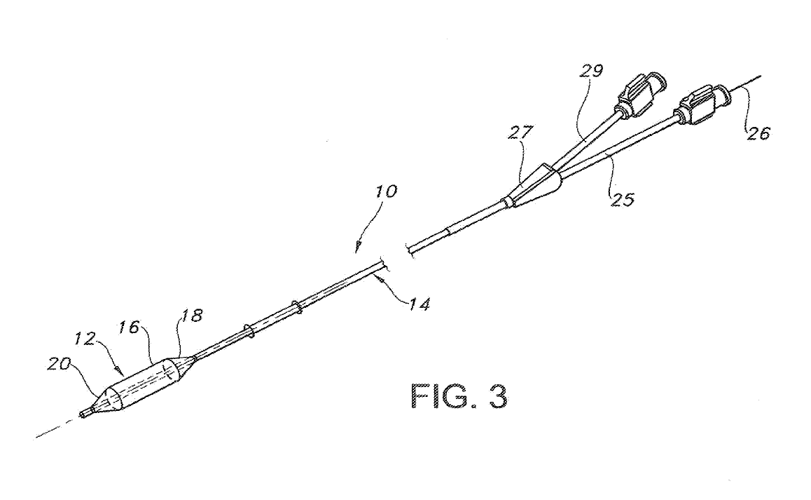

Provided is a catheter 10 having a distal portion 11 with a balloon 12 mounted on a catheter tube 14. Referring to FIGS. 1, 2, and 3, the balloon 12 has an intermediate section 16, or “barrel” having the working surface W, and end sections 18, 20. In one embodiment, the end sections 18, 20 reduce or taper in diameter to join the intermediate section 16 to the catheter tube 14 (and thus sections 18, 20 are generally termed cones or cone sections). The balloon 12 is sealed to catheter tube 14 at balloon ends (proximal 15a and distal 15b) on the end sections 18, 20 to allow the inflation of the balloon 12 via one or more inflation lumens 17 extending within catheter tube 14 and communicating with the interior of the balloon 12.

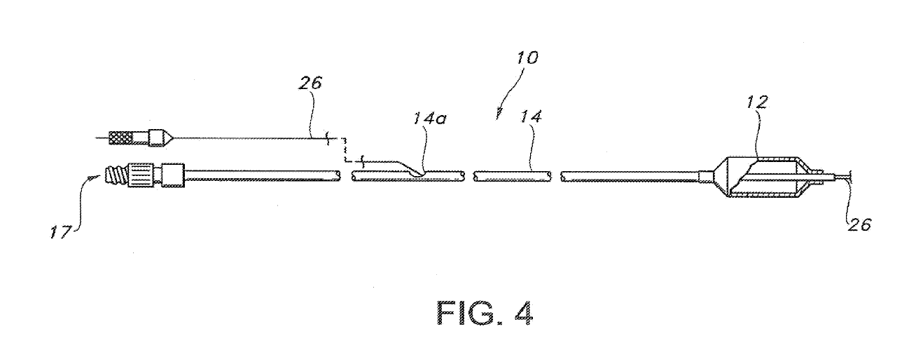

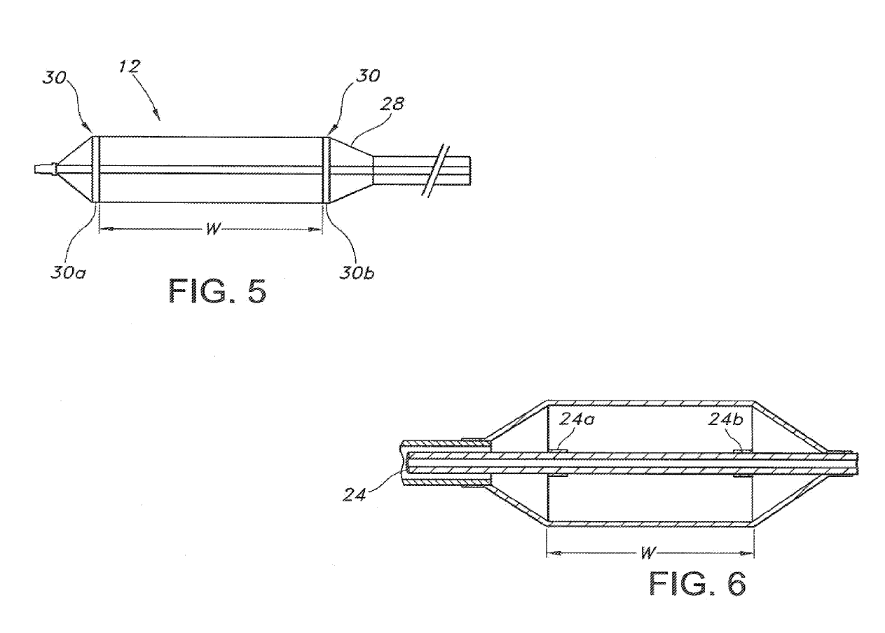

The catheter tube 14 also includes an elongated, tubular shaft 24 forming a guidewire lumen 23 that directs the guidewire 26 through the catheter 10. As illustrated in FIG. 3, this guidewire 26 may be inserted through a first port 25 of a connector, such as a hub 27, into the lumen 23 to achieve an “over the wire” (OTW) arrangement, but could also be provided in a “rapid exchange” configuration in which the guidewire 26 enters the lumen through a lateral opening 14a closer to the distal end (see FIG. 4). A second port 29 may also be associated with catheter 10, such as by way of connector 27, for introducing a fluid (e.g., saline, a contrast agent, or both) into the interior of the balloon 12 via the inflation lumen 17.

Balloon 12 may include a single or multi-layered balloon wall 28. The balloon 12 may be a non-compliant balloon having a balloon wall 28 that maintains its size and shape in one or more directions when the balloon is inflated. The balloon 12 in such case also has a pre-determined surface area that remains constant during and after inflation, also has a pre-determined length and pre-determined circumference that each, or together, remain constant during and after inflation. However, the balloon 12 could be semi-compliant or compliant instead, depending on the particular use.

In order to provide for enhanced locatability during an interventional procedure, the catheter 10 may have a radiopaque quality. In one embodiment, this radiopaque quality is provided in a manner that allows for a clinician to differentiate, with relative ease and high precision, one portion of the balloon 12 from another (such as the barrel section 16 including the working surface W from the cone sections 18, 20). This helps the clinician ensure the accurate positioning of the balloon 12 and, in particular, the working surface W, at a specified treatment area. This may be especially important in the delivery of a particular item, such as a drug or stent, via the balloon working surface W, as outlined in more detail in the following description.

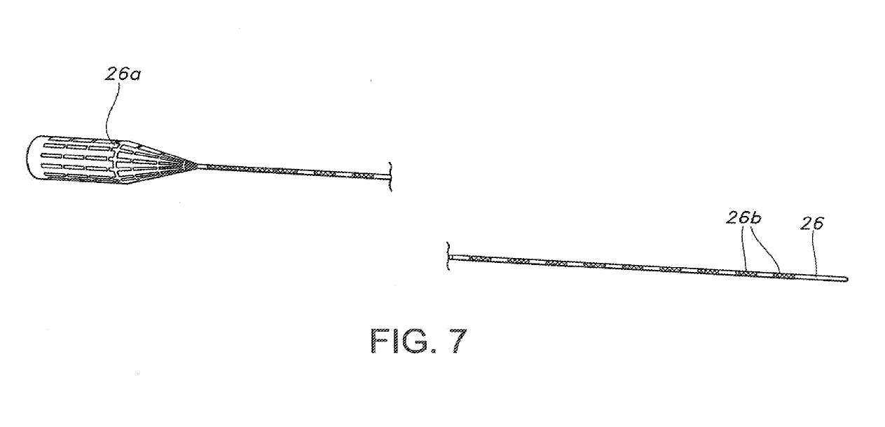

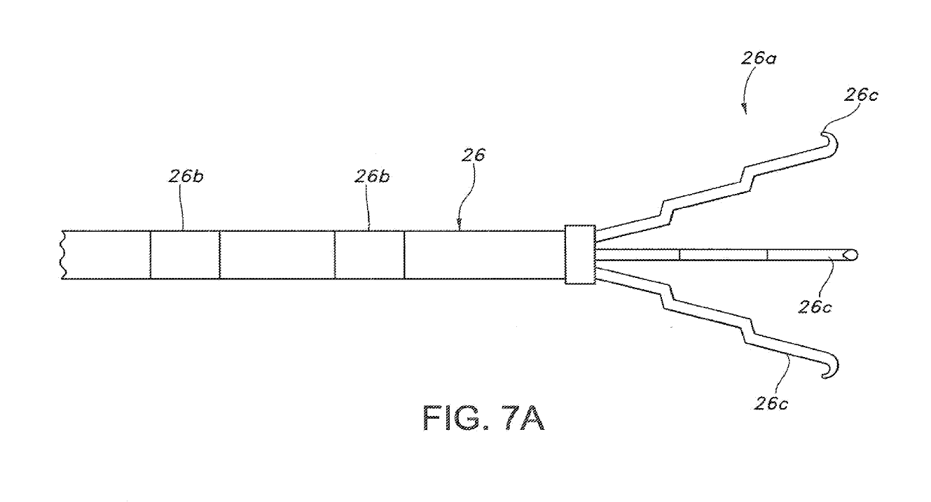

In one embodiment, the radiopaque quality is achieved by providing or strategically positioning one or more at least partially radiopaque identifiers or markings 30. The marking 30 may be provided at one or more locations on the balloon wall 28 (either on it or within it), such as in the form of one or more bands 30a, 30b, to create a defined portion as the working surface W, as shown in FIG. 5. Alternatively, the markings 30 may be provided on the catheter shaft 24 at positions corresponding to locations on the balloon 12. For example, the marking 30 may comprise one or more bands 24a, 24b on the shaft 24 positioned so as to correspond to the ends of the working surface W when the balloon 12 is inflated, as shown in FIG. 6.

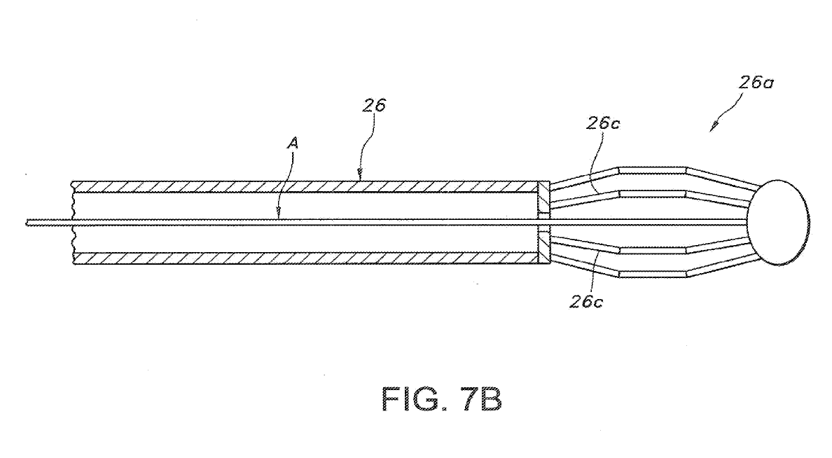

In accordance with one aspect of the disclosure, and as shown in FIG. 7, the guidewire 26 is specially adapted for use in avoiding the problem of geographic misalignment. In the illustrated embodiment, the guidewire 26 includes an anchor 26a, and one or more markings 26b, which may be radiopaque. The anchor 26a may be positioned at the distal end of the guidewire 26 for anchoring the guidewire in position within the vasculature following insertion. The one or more markings 26b may be provided proximally of the anchor 26a and, in the case of multiple markings, at spaced intervals along the length of the guidewire 26.

The anchor 26a may be any device suitable for anchoring the guidewire 26 in the vasculature. For instance, the anchor 26a may comprise a stent, such as for example, a self-expanding stent, a self-expanding braided stent, or other an expandable device (e.g., balloon or other anchor). In the case of a self-expanding stent, the device may be made from a shape memory material, such as Nitinol, which is then automatically expanded at the desired location to provide the anchoring function.

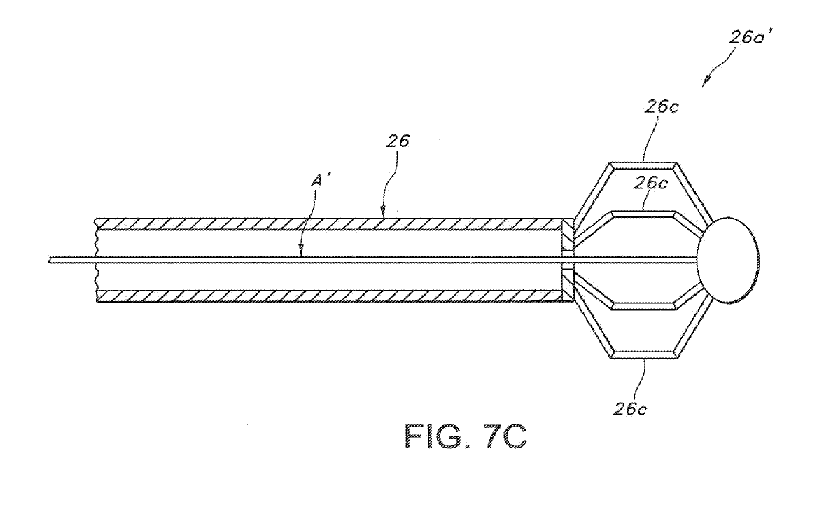

If a balloon or like inflatable device serves as the anchor 26a, the guidewire 26 may include a lumen for supplying an inflation fluid to the interior of the balloon in order to actuate it and create the desired anchoring function for the guidewire. Other possible examples include an anchor 26a with a plurality of radially extensible fingers 26c for being expanded in the vasculature (FIG. 7a), either as a result of an automatic shape change (such as via shape memory material) or actuation (note actuator A in FIG. 7b, and compare expanded condition of anchor 26a′ in FIG. 7c, with actuator A′ drawn in the proximal direction). A simple barb could also be used as the anchor 26a, as shown in FIG. 8. The particular version of anchor is not considered important, as long as sufficient force can be provided for retaining the guidewire 26 in a desired position in the vasculature.

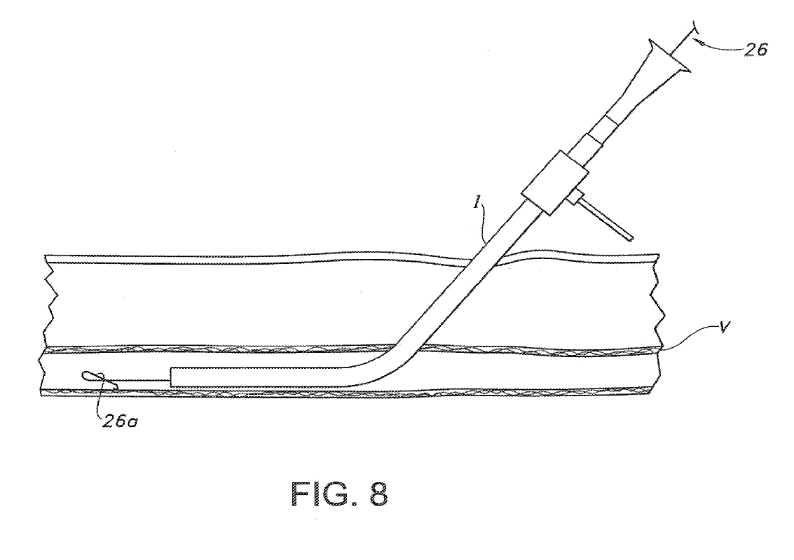

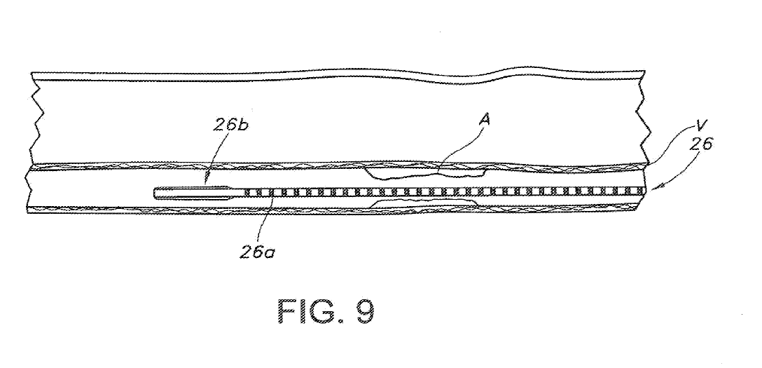

As noted above, and with reference to FIGS. 8-10, the at least partially radiopaque guidewire 26 may be positioned within a vessel V, as such as by using a device called an introducer I. Under fluoroscopy, the portion of the wire 26 with the markings 26b is positioned in the treatment area A, and the anchor 26a is deployed (which again may be the barb anchor shown in FIG. 8, or the expandable anchor shown in FIGS. 9-10). The positioning may be such that one or more of the radiopaque markings 26b align with the treatment area A in a predetermined manner, or alternatively, the location of the treatment area A relative to the markings 26b may be noted by the clinician (such as by counting the markings corresponding to the treatment area or using known distances of or between them to assess distances in the vasculature).

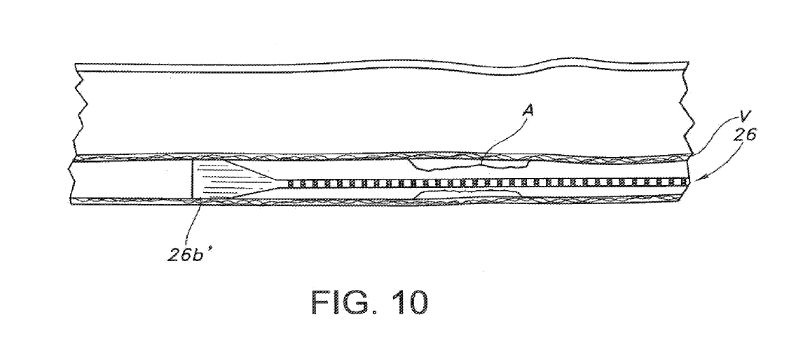

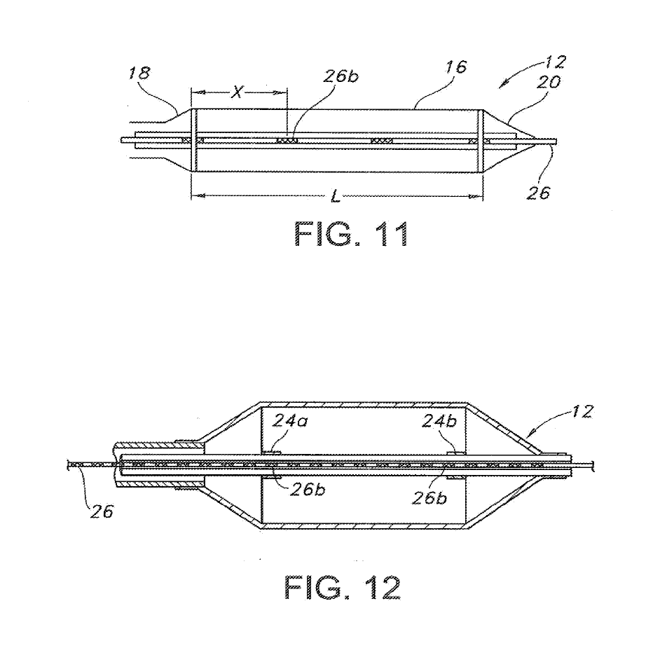

A catheter 10 with radiopaque markings, such as the marker bands 24a, 24b on the shaft 24 (FIG. 11) or markings 30 on the balloon 12 (FIG. 12), may then be co-located with the guidewire 26 such that the various radiopaque markings 26b, 30 correspond to each other, such as by aligning. As should be appreciated, the guidewire 26 may include regular markings 26b, but irregular markings could be provided as well. The distance between the markings on the catheter 10, such as bands 24a, 24b on the shaft 24 or bands 30a, 30b on the balloon 12, may also be an integer multiple of the distance between the markings 26b on the guidewire 26 (e.g., 3 x is approximately equal to L in FIG. 11, where x is the pitch of the markings 26b, and L is the length between the markings 30 on the catheter 10), such that the working surface W registers in alignment with the treatment area A when it corresponds to the markings 26b (which may be effected by selecting a guidewire with at least one pair of markings 26b spaced at a distance that corresponds to the estimated treatment area size for a given patient or procedure). In any case, alignment with the markings 30 on the balloon 12 may be easily achieved, as indicated in FIGS. 11 and 12.

As a result of this approach, improved alignment is assured, which may help to avoid the geographic misalignment between the balloon 12 and the treatment area A. This is especially true during the positioning of a second balloon including a treatment at the treatment area A, such as during a second intervention, since the guidewire 26 will remain at the pre-positioned location, as will the radiopaque markings 26b. Accordingly, a high degree of repeatability is also afforded, which is especially important when the treatment afforded by the second balloon is a pharmaceutical composition.

The radiopaque markings 26b of the guidewire 26 may be provided in a variety of ways, but should not compromise the desired flexibility typically afforded. They may be formed as integral parts of the wire, or may be separately attached (including by bonding, winding (e.g., a spring), coating, or like processes). Specific examples include forming the wire 26 by winding a highly radiopaque winding wire of platinum, gold, or tungsten about a central core wire, applying a radiopaque ink to the wire, bonding a radiopaque sleeve to the wire, such as a tungsten filled polymer sleeve, or affixing a series of small radiopaque metal bands to the wire. The markings 26b may be provided as radiopaque portions of the wire 26 interposed with non-radiopaque portions, or the markings 26b may comprise radiopaque portions of the wire 26 that have a different radiopaque quality as compared to other portions of the wire.

Examples of radiopaque materials include, but are not limited to, finely divided tungsten, tantalum, bismuth, bismuth trioxide, bismuth oxychloride, bismuth subcarbonate, other bismuth compounds, barium sulfate, tin, silver, silver compounds, rare earth oxides, and many other substances commonly used for X-ray absorption. The amount used may vary depending on the desired degree of radiopacity.

The balloon 12 with markings 30 may be created in various ways. For example, the markings 30 may be provided by applying a radiopaque material to a surface of the balloon wall 28 at the desired location in the form of a coating. This may be done by inking, spraying, printing, stamping, painting, adhering, or otherwise depositing (such as by chemical vapor deposition) the radiopaque material onto the balloon wall 28 (possibly with the application of a mask or the like, in which case the techniques of dipping or rolling the balloon 12 in the radiopaque material to form the desired coating could be used). The marking 30 may be provided during the process for fabricating the balloon wall 28, such as for example during a coextrusion or blow molding process.

The marking 30 may also comprise a radiopaque material applied to the interior surface of the balloon wall 28, such as by painting or other bonding. In one example, the radiopaque material comprises gold applied to the exterior or interior surface of the balloon 12, such as in the form of a band (which may be any of the bands described herein). The gold may be applied in leaf form, given its softness and malleability, which also means that it will not in any way hinder the expansion of the balloon 12.

Any catheter, balloon, or guidewire according to the disclosure may be designed to deliver a payload (such as a therapeutic agent (e.g., a drug, such as paclitaxel, rapamycin, heparin and the like), a stent, a stent graft, or a combination thereof) or a working element (such as a cutter, focused force wire, or the like) to a specified location within the vasculature.

While the disclosure presents certain embodiments to illustrate the inventive concepts, numerous modifications, alterations, and changes to the described embodiments are possible without departing from the sphere and scope of the present invention, as defined in the appended claims. Any ranges and numerical values provided in the various embodiments are subject to variation due to tolerances, due to variations in environmental factors and material quality, and due to modifications of the structure and shape of the balloon, and thus can be considered to be approximate and the term “approximately” means that the relevant value can, at minimum, vary because of such factors. Also, the drawings, while illustrating the inventive concepts, are not to scale, and should not be limited to any particular sizes or dimensions. Accordingly, it is intended that the present disclosure not be limited to the described embodiments, but that it has the full scope defined by the language of the following claims, and equivalents thereof.

1. An apparatus for treating a treatment area in the vasculature using a catheter including a working surface having a length, comprising:
a guidewire for guiding the catheter to the treatment area and including an anchor for selectively anchoring the guidewire to the vasculature, said guidewire including at least one radiopaque marking for corresponding to the length of the working surface of the catheter when positioned at the treatment area.

2. The apparatus of claim 1, wherein the guidewire includes a plurality of radiopaque markings corresponding to the length of the working surface of the catheter.

3. The apparatus of claim 2, wherein the plurality of radiopaque markings on the guidewire are equidistantly spaced.

4. The apparatus of claim 2, wherein the plurality of radiopaque markings are irregularly spaced.

5. The apparatus of claim 1, wherein the anchor is selected from the group consisting of a self-expanding stent, a self-expanding braided stent, a barb, a plurality of extensible fingers, and an inflatable balloon.

6. The apparatus of claim 5, wherein the anchor comprises an inflatable balloon and the guidewire comprises a lumen for inflating the balloon.

7. The apparatus of claim 5, wherein the anchor comprises a shape memory material.

8. The apparatus of claim 1, wherein the anchor is provided at a distal end of the guidewire, and the at least one second radiopaque marking is proximal of the anchor.

9. An apparatus for treating a treatment area in the vasculature, comprising: a catheter including at least one first radiopaque marking identifying a working surface; and a guidewire for guiding the catheter to the treatment area and including an anchor for anchoring the guidewire to the vasculature, said guidewire including at least one second radiopaque marking adapted for corresponding to the at least one first radiopaque marking of the catheter when positioned at the treatment area.

10. The apparatus of claim 9, wherein the guidewire includes a plurality of second radiopaque markings.

11-12. (canceled)

13. The apparatus of claim 9, wherein the anchor is selected from the group consisting of a self-expanding stent, a self-expanding braided stent, a barb, a plurality of extensible fingers, and an inflatable balloon.

14. The apparatus of claim 13, wherein the anchor comprises an inflatable balloon and the guidewire comprises a lumen for inflating the balloon.

15. The apparatus of claim 9, wherein the anchor is provided at a distal end of the guidewire, and the at least one second radiopaque marking is proximal of the anchor.

16. The apparatus of claim 9, wherein the catheter includes an inflatable balloon having the working surface.

17. The apparatus of claim 16, wherein the inflatable balloon includes the at least one radiopaque marking.

18. The apparatus of claim 9, wherein the catheter comprises a shaft adapted for receiving the guidewire, said shaft further including the at least one first radiopaque marking for aligning with the at least one second radiopaque marking of the guidewire.

19. The apparatus of claim 9, further including a second catheter including an expandable device having a treatment.

20. The apparatus of claim 19, wherein the treatment is selected from the group consisting of a drug, a stent, a stent graft, a cutter, a focused force wire, or any combination of the foregoing.

21. A kit for treating a treatment area in the vasculature, comprising:
a first catheter including at least one first radiopaque marking; a second catheter including at least one second radiopaque marking; and a guidewire for guiding the first or second catheter to the treatment area and including an anchor for anchoring the guidewire to the vasculature, said guidewire including at least one third radiopaque marking adapted for corresponding to the at least one first radiopaque marking of the first catheter or the at least one second radiopaque marking of the second catheter when positioned at the treatment area.

22. The apparatus of claim 1, further comprising an actuator or actuating means, the actuator or actuating means being arranged to actuate the anchor to thereby anchor the guidewire to the vasculature.

23. The apparatus of claim 1, the anchor being arranged so that it will, when introduced to a location into a patient's vasculature, automatically assume a configuration capable of anchoring the guidewire.

24-29. (canceled)

30. The apparatus of claim 16, wherein the inflatable balloon includes one first radiopaque marking at one end of the working surface and another first radiopaque marking at a second end of the working surface.

31. The apparatus of claim 9, wherein the catheter includes one first radiopaque marking at one end of the working surface and another first radiopaque marking at a second end of the working surface.

32. The apparatus of claim 16, wherein the inflatable balloon includes a drug.
